(19) United States
(12) Patent Application Publication
(10) Pub. No.: US 2025/0000452 A1
(43) Pub. Date: Jan. 2, 2025

(54) COMPUTERIZED SYSTEM AND METHOD FOR DYNAMIC SLEEP MANAGEMENT

(71) Applicant: PLUME DESIGN, INC., Palo Alto, CA (US)

(72) Inventor: Christopher MINTON, Cedar Park, TX (US)

(21) Appl. No.: 18/342,208

(22) Filed: Jun. 27, 2023

(51) Int. Cl. A61B 5/00

(52) U.S. Cl. CPC A61B 5/4815; A61B 5/6802; A61B 5/742

(57) ABSTRACT

Disclosed are systems and methods that provide a novel framework for personalized sleep management for a user. The framework can provide dynamically determined sleep data for a user, determined from data collected from the sensor(s) of device(s) in a location of a user, that is correlated with sleep data determined in a same or similar manner for other users (e.g., users who share a demographic with the user). The framework provides a comprehensive sleep optimization system that provides personalized insights and recommendations to aid and/or effectuate users achieving a restful and rejuvenating sleep.

### FIELD OF THE DISCLOSURE

The present disclosure is generally related to a sleep monitoring and analytics system, and more particularly, to a computerized framework for computer-generated sleep insight based on sleeping patterns extracted across different populations.

### BACKGROUND

In an age in which a range of sleep tracking devices are widely available, many individuals seek accurate and informative measures of their sleep quality. However, conventional devices fail to capture (1) data relating to life choices that impact sleep quality and (2) data that puts an individual's measure of sleep quality into context.

### SUMMARY OF THE DISCLOSURE

The disclosed systems and methods, as described herein, provide a novel computerized framework that provides dynamically determined sleep data for a user that is correlated with the sleep data of other users (e.g., users who share a demographic with the user). According to some embodiments, the disclosed systems and methods provide a comprehensive sleep optimization system that delves deep into the factors that influence the sleep quality of users in different groups (e.g., groups defined by user demographic).

According to some embodiments, the disclosed framework can (1) trigger a sensor of a sleep-tracking device (e.g., a wearable device) to collect data for a user, (2) generate a measure of the user's sleep based on the collected data, (3) determine a demographic of the user and a correlation between the measure of the user's sleep and measures of user sleep generated for other uses associated with the demographic, and/or (4) communicate, over a network, the correlation to the user (e.g., via a digital display presented via a display element of a user device). In some examples, the disclosed framework can also (1) identify a subset of the users associated with the demographic who have a measure of user sleep that falls below a threshold value, (2) receive (e.g., as an output from a trained model) a feature associated with such users, and (3) communicate, over the network via the digital display, the feature to the user as part of a sleep-improvement suggestion (e.g., in response to determining that the user's measure of sleep falls below the threshold and/or that the feature is associated with the user). In certain examples, the disclosed framework may identify the feature by (1) identifying, for each particular user within the subset, one or more devices within a location associated with the particular user. (2) collecting data from the identified devices, (3) inputting the data collected from the identified device to the trained model, and (4) receiving the feature as an output from the trained model (e.g., generated by the trained model based on the inputted data). The feature may represent any computer-detectable feature. Examples of the feature may include, without limitation, an environmental feature (e.g., a geographic region, an altitude of a geographic region, a measure of pollution for a geographic region, a population size of a geographic region, etc.), a pattern of activity, such as a pattern of screen usage and/or movement (e.g., determined from data collected by devices, within a location of the user, with sensor capabilities for tracking activities such as movement), and/or a biometric feature (e.g., a weight, blood oxygen level, a temperature level, a heartrate, and/or a blood pressure of the user). Each of these aspects of the disclosed framework will be discussed in more detail below in relation to at least FIGS. 1 and 3.

According to some embodiments, a method is disclosed for a DI-based computerized framework (e.g., for performing one or more of the steps discussed in connection with FIG. 3). In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for automatically and dynamically managing real-time sleep activity of a user.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary workflow according to some embodiments of the present disclosure;

FIG. 4 is a block diagram illustrating a computing device showing an example of a client or server device used in various embodiments of the present disclosure.

FIG. 5 depicts an exemplary implementation of an architecture according to some embodiments of the present disclosure; and

FIG. 6 depicts an exemplary implementation of an architecture according to some embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a net worked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2^{nd}, 3^{rd}, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, a smart ring, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, as discussed in more detail below, the disclosed framework can function/operate to provide personalized insights and/or recommendations to users based on (1) their own specific/unique data and/or (2) how their own specific/unique data compares to aggregated data for a population of the user. Thus, a tailored approach to sleep insight and/or improvement can be compiled and automatically provided to the user, which can be provided upon request and/or a determined time (e.g., a time a user is deemed to be about to sleep and/or to have just woken up). In some embodiments, the disclosed framework can leverage such insights, in addition to the recommendations to the users, to control the location in which they are sleeping. For example, access points, devices (e.g., televisions, smart phones, and the like), lights and/or any other smart home feature can be engaged and/or toggled to different and/or modified modes upon the determination of a user's sleep, which can assist the user from engaging in activity that may be detrimental to their known sleep habits.

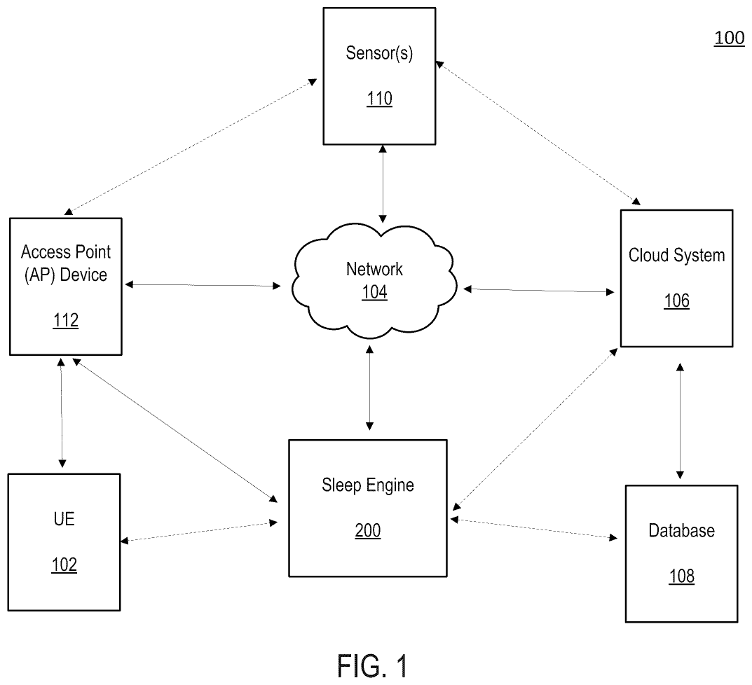

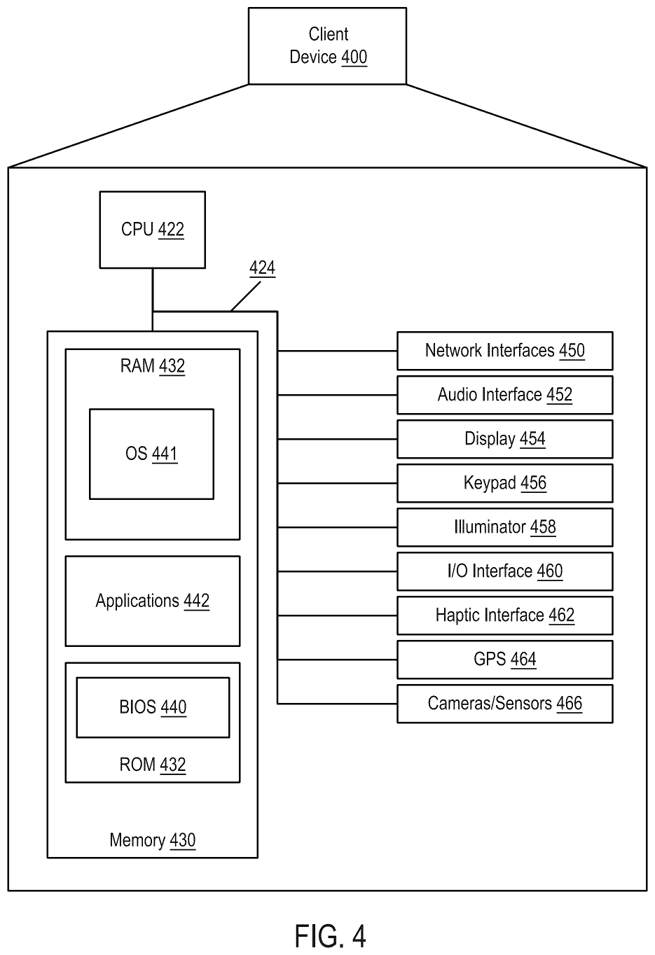

With reference to FIG. 1, system 100 is depicted which includes user equipment (UE) 102 (e.g., a client device, as mentioned above and discussed below in relation to FIG. 4), access point (AP) device 112, network 104, cloud system 106, database 108, sensors 110 and sleep engine 200. It should be understood that while system 100 is depicted as including such components, it should not be construed as limiting, as one of ordinary skill in the art would readily understand that varying numbers of UEs, AP devices, peripheral devices, sensors, cloud systems, databases and networks can be utilized; however, for purposes of explanation, system 100 is discussed in relation to the example depiction in FIG. 1.

According to some embodiments, UE 102 can be any type of device, such as, but not limited to, a mobile phone, tablet, laptop, sensor, IoT device, autonomous machine, and any other device equipped with a cellular or wireless or wired transceiver. For example, UE 102 can be a smart ring, which as discussed below in more detail, can enable the identification and/or collection of vitals of the wearing user. In some embodiments, such vitals can correspond to, but not be limited to, heart rate, heart rate variability (HRV), blood oxygen levels, blood pressure, hydration temperature, pulse, motion, sleep, and/or any other type of biometric for a person, or some combination thereof.

In some embodiments, a peripheral device (not shown) can be connected to UE 102, and can be any type of peripheral device, such as, but not limited to, a wearable device (e.g., smart ring or smart watch), printer, speaker, sensor, and the like. In some embodiments, the peripheral device can be any type of device that is connectable to UE 102 via any type of known or to be known pairing mechanism, including, but not limited to, WiFi, Bluetooth™, Bluetooth Low Energy (BLE), NFC, and the like. For example, the peripheral device can be a smart ring that connectively pairs with UE 102, which is a user's smart phone.

According to some embodiments, AP device 112 is a device that creates a wireless local area network (WLAN) for the location. According to some embodiments, the AP device 112 can be, but is not limited to, a router, switch, hub and/or any other type of network hardware that can project a WiFi signal to a designated area. For example, an AP device 112 can be a Plume Pod™, and the like. In some embodiments, UE 102 may be an AP device.

According to some embodiments, sensors 110 (or sensor devices 110) can correspond to any type of device, component and/or sensor associated with a location of system 100 (referred to, collectively, as “sensors”). In some embodiments, the sensors 110 can be any type of device that is capable of sensing and capturing data/metadata related to a user and/or activity of the location. For example, the sensors 110 can include, but not be limited to, cameras, motion detectors, door and window contacts, heat and smoke detectors, passive infrared (PIR) sensors, time-of-flight (ToF) sensors, and the like.

In some embodiments, the sensors 110 can be associated with devices associated with the location of system 100, such as, for example, lights, smart locks, garage doors, smart appliances (e.g., thermostat, refrigerator, television, personal assistants (e.g., Alexa®, Nest®, for example)), smart rings, smart phones, smart watches or other wearables, tablets, personal computers, and the like, and some combination thereof. For example, the sensors 110 can include the sensors on UE 102 (e.g., smart phone) and/or peripheral device (e.g., a paired smart watch). In another example, sensors 110 can correspond to the sensors on a user's smart ring.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service provider and/or network provider from where services and/or applications may be accessed, sourced or executed from. For example, system 106 can represent the cloud-based architecture associated with a smart home or network provider, which has associated network resources hosted on the internet or private network (e.g., network 104), which enables (via engine 200) the sleep management discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, AP device 112, sensors 110, and the services and applications provided by cloud system 106 and/or sleep engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

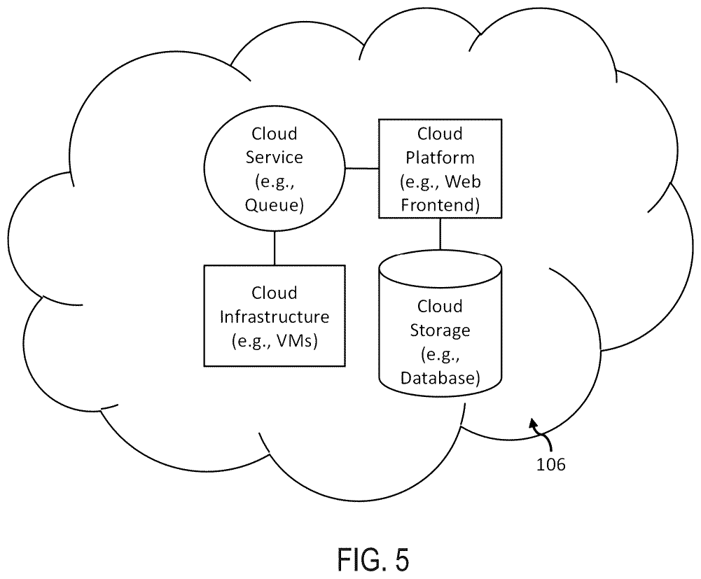

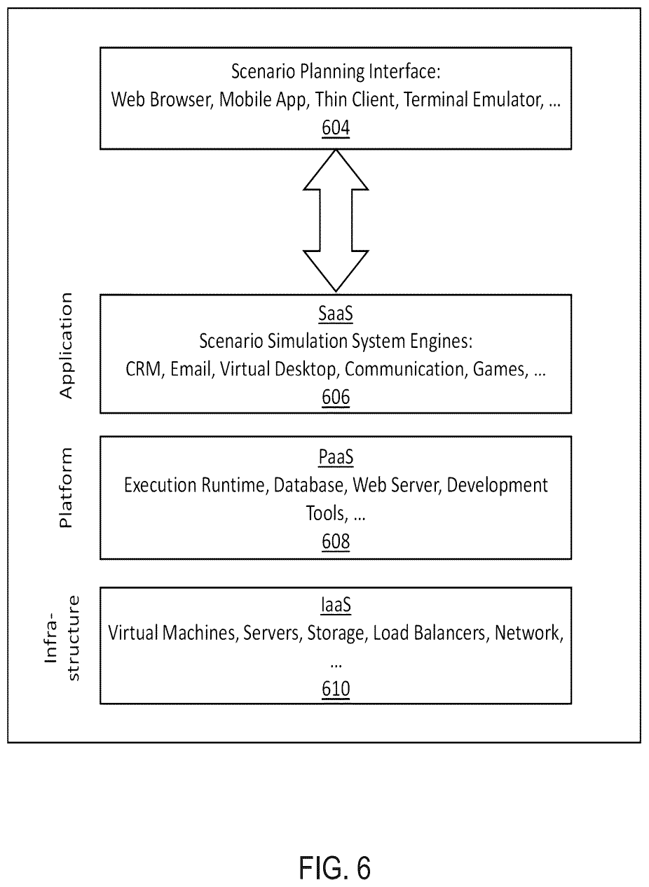

Turning to FIGS. 5 and 6, in some embodiments, the exemplary computer-based systems/platforms, the exemplary computer-based devices, and/or the exemplary computer-based components of the present disclosure may be specifically configured to operate in a cloud computing/architecture 120 such as, but not limiting to: infrastructure as a service (IaaS) 610, platform as a service (PaaS) 608, and/or software as a service (SaaS) 606 using a web browser, mobile app, thin client, terminal emulator or other endpoint 604. FIGS. 5 and 6 illustrate schematics of non-limiting implementations of the cloud computing/architecture(s) in which the exemplary computer-based systems for administrative customizations and control of network-hosted application program interfaces (APIs) of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL).

According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository

Sleep engine 200, as discussed above and further below in more detail, can include components for the disclosed functionality. According to some embodiments, sleep engine 200 may be a special purpose machine or processor and can be hosted by a device on network 104, within cloud system 106, on AP device 112 and/or on UE 102. In some embodiments, engine 200 may be hosted by a server and/or set of servers associated with cloud system 106.

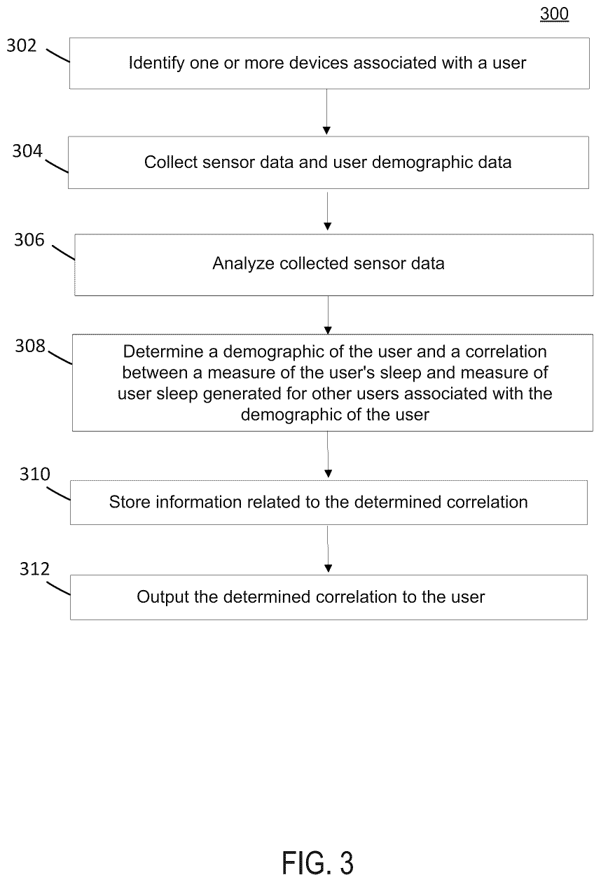

According to some embodiments, as discussed in more detail below, sleep engine 200 may be configured to implement and/or control a plurality of services and/or microservices. In some examples, each of the plurality of services/microservices are configured to execute a plurality of workflows associated with performing the disclosed sleep management. Non-limiting embodiments of such workflows are provided below in relation to at least FIGS. 3-4.

According to some embodiments, as discussed above, sleep engine 200 may function as an application provided by cloud system 106. In some embodiments, sleep engine 200 may function as an application installed on a server(s), network location and/or other type of network resource associated with system 106. In some embodiments, sleep engine 200 may function as an application installed and/or executing on UE 102 and/or sensors 110 (and/or AP device 112, in some embodiments). In some embodiments, such application may be a web-based application accessed by AP device 112, UE 102 and/or devices associated with sensors 110 over network 104 from cloud system 106. In some embodiments, sleep engine 200 may be configured and/or installed as an augmenting script, program or application (e.g., a plug-in or extension) to another application or program provided by cloud system 106 and/or executing on AP device 112, UE 102 and/or sensors 110.

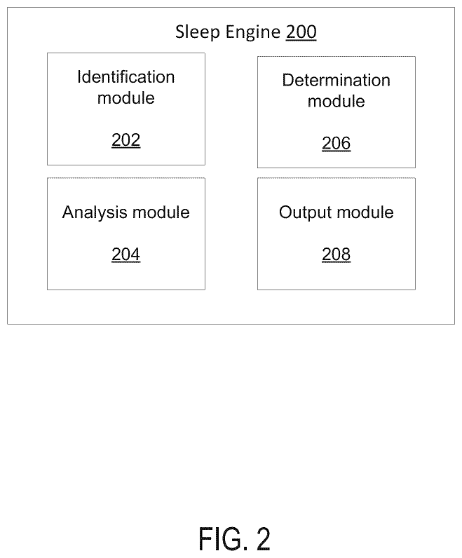

As illustrated in FIG. 2, according to some embodiments, sleep engine 200 includes identification module 202, analysis module 204, determination module 206 and output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 3, Process 300 provides non-limiting example embodiments for the disclosed computerized sleep management framework. According to some embodiments, Process 300 provides non-limiting embodiments for (1) determining (e.g., via sleep engine 200) user data (e.g., sleep data, demographic data, biometric data, patterns of behavior, and/or environmental data) and/or (2) digitally controlling, managing and/or manipulating (e.g., via sleep engine 200) the operational status of a location. The leveraged and stored information can be utilized to optimize a user's sleep.

According to some embodiments, Steps 302-304 of Process 300 can be performed by identification module 202 of sleep engine 200; Step 306 can be performed by analysis module 204; Step 308 can be performed by determination module 204; and Steps 310 and 312 can be performed by output module 208.

According to some embodiments, Process 300 begins with Step 302 where a set of devices associated with a user (and in some embodiments, associated with a location, for example, a user's home) are identified. According to some embodiments, the devices can be associated with any type of UE 102, AP device 112, sensors 110, and the like, discussed above in relation to FIG. 1. For example, the devices can at least include, but are not limited to, a sleep-tracking device (e.g., a wearable device such as a user's smart watch and/or smart ring that they wear daily), the user's smart phone, tablet devices in the home connected to the local network (e.g., Wi-Fi), televisions, light sensors, door sensors, routers/modems providing the network for the location, and the like. Additional, non-limiting examples of sensors and the types of collectable data are discussed above at least in relation to FIG. 1.

In some embodiments, the identified devices can be paired and/or connected with another device (e.g., sensor 110, engine 200 and/or UE 102) via a cloud (e.g., Plume® cloud, for example) and/or cloud-to-cloud (C2C) connection (e.g., establish connection with a third-party cloud, which connects with cloud system 106, for example).

In Step 304, sleep engine 200 can operate to trigger the identified devices to collect data about the user (e.g., referred to as user data). This user data can include any type or form of data relating to a user and/or an environment (e.g., a location and/or dwelling) of the user (e.g., any data that can be collected from a device identified in Step 302). In some examples, the user data can represent or include sensor data (e.g., collected via sensor(s) 110). According to some embodiments, the user data can be collected continuously and/or according to a predetermined period of time or interval. For example, sleep engine 200 can periodically ping each or a portion of the devices for a reply and/or can push and/or fetch protocols to collect user data from each sensor. Additionally or alternatively, the user data may be collected based on detected events. In some embodiments, a type and/or quantity of user data collected may be directly tied to the type of device performing such data collection. For example, a sensor associated with lights within the user's bedroom may only collect user data when a light in that room is turned on (and/or turned off from previously being turned on). In this example, user data can provide, but is not limited to, an on event, which can indicate, but is not limited to, the identity of the light/room, time of toggling, duration of being turned on, frequency of being turned on, and the like, or some combination thereof. In another non-limiting example, a gyroscope sensor on a user's smartphone and/or smart ring can detect when a user is moving, the type and/or metrics of such movements, etc.

According to some embodiments, the user data can be generated (e.g., derived) from data collected from UE 102. For example, as discussed above (e.g., in examples in which user data is collected from a wearable device such as a smart ring), the user data can provide biometrics (e.g., vitals) for the user, which can include information related to, but are not limited to, heart rate, HRV, blood oxygen levels, blood pressure, hydration, body temperature, pulse, motion, and the like, or some combination thereof. In some such examples, the biometrics, inclusive of the heart rate, HRV, blood pressure and blood oxygen level information, for example, can provide insights into a user's cardiovascular health, respiratory health, and/or fitness level. Additionally or alternatively, the biometrics can further provide or contribute to tracked information related to the user's physical activity, including, but not limited to, the number of steps taken, distance traveled, and calories burned, and the like. As another example, the biometrics can be used to derive data relating to the user's stress levels and/or to detect potential illness.

In some examples, the user data can include sleep data (e.g., biometric data, associated with sleep, collected by a sensor of a sleep-tracking device operating with UE 102). In these examples, sleep engine 200 may detect, for a user, a measure of the user's sleep generated (e.g., derived) based on the sleep data (e.g., by triggering the sensor to collect the sleep data and then generating the measure of the user's sleep based on the sleep data). The measure of the user's sleep can include one or more of any type or form of measurement (value or metric) related to sleep (e.g., sleep quantity and/or quality). Measures relating to sleep can include, but are not limited to, measures relating to sleep latency (e.g., time it takes for the user to fall asleep or into a deep sleep, and the like), total sleep duration, deep sleep duration (e.g., length of time spent in restorative sleep), light sleep duration (e.g., time spent in lighter sleep stages), restlessness (e.g., frequency and/or degree of movement during sleep and/or when lying in bed), nighttime awakenings (e.g., number of times the user wakes up and/or gets out of bed), and the like or some combination thereof.

In addition to collecting user sleep data, in some examples the devices identified at Step 302 may collect sleep-relevant data. Sleep-relevant data may represent data that is not indicative of an amount, type, and/or quality of sleep but that may be associated with (e.g., may influence) the amount, type, and/or quality of sleep experienced by a user. Examples of sleep-relevant data may include, without limitation, environmental data (e.g., data relating to a location such as a home and/or a geographic area associated with a user), data relating to a user pattern of activity (e.g., a type of activity engaged in by a user and/or a frequency and/or timing of an activity), and/or data relating to a biometric. In some embodiments, the sleep-relevant data can be based on lifestyle factors (e.g., factors inferred from sensor data collected from sensors 110 and/or UE 102). Such lifestyle factors can include, but are not limited to, screen/Internet use (e.g., time spent on screens before bed and/or throughout the day), type of network activity (e.g., gaming, social media, streaming, and the like), exercise patterns (e.g., timing, duration, and/or intensity of physical activity during the day), motion detection and/or user location (e.g., motion data in/around and/or outside the location during the day and/or around bedtime), diet and nutrition, and the like, or some combination thereof. In some examples, all data that may be collected by the devices identified at Step 302 can be considered (e.g., designated) as potentially sleep-relevant. In one such example, any (e.g., all) data collected by such devices may be inputted to a trained model (e.g., as part of the analyzing that will be described shortly in connection with step 306) and sleep-relevant data may be received as an output from the trained model. In this example, the trained model may detect features (e.g., user features, such as user behaviors and/or health metrics, and/or features relating to an environment of the user such as the user's home, the street, neighborhood, city, and/or county the user lives in, etc.) that affect sleep that users or researchers may be surprised to learn are associated with sleep.

In some embodiments, the user data collected by the devices may include user information submitted by the user. Such information may include, without limitation, social information (e.g., contacts submitted by the user), health information (e.g., a health condition submitted by the user), user activity reported by the user (e.g., a submission of foods consumed by the user), a mental health status (e.g., a submission of a level of stress indicated by the user), height information, weight information, BMI information, etc.

In some examples, the user information submitted by the user may include demographic information (e.g., a user submission of a demographic associated with the user). A demographic associated with a user may include, without limitation, an age, a gender, an occupation, a level of education, a family status, an ethnicity, a location (e.g., associated with the user's home and/or work), a biometric (e.g., a weight, a BMI score, etc.) and/or a culture. In addition (or as an alternative to) detecting demographic information submitted by a user, in some examples, some or all demographic information (e.g., geographic information and/or information derivable from biometric data) may be collected by the devices identified in Step 302 and/or derived from data that is collected by the devices.

The disclosed sleep management system may enable a user to submit user information in a variety of ways. For example, sleep engine 200 can (1) generate a user interface display that includes elements for digitally submitting user information, (2) trigger a user device (e.g., included as part of UE 102) to present the user interface display via a display element of the user device, and/or (3) receive user information as user input submitted to the elements presented within the user interface display. Sleep engine 200 may trigger the user device to present the user interface display in a variety of digital contexts (e.g., as part of a registration process, as input to a health tracking application, as input to a sleep management application, etc.).

In some embodiments, user data may be derived and/or mined from stored user data within an associated or third-party cloud. For example, sleep engine 200 can be associated with a cloud, which can store collected network traffic and/or collected user data for the user in an associated account of the user. Thus, in some embodiments, Step 304 can involve querying the cloud for information about the user, which can be based on a criteria that can include, but is not limited to, a time, date, activity, event, other collected user data, and the like, or some combination thereof.

In some embodiments, the collected user data in Step 304 can be stored in database 108 in association with an identifier (ID) of a user, an ID of the location and/or an ID of an account of the user/location. In some examples, database 108 may also include information relating (e.g., relevant) to the user data. For example, in embodiments in which a user's user data includes a location of a home of the user, database 108 may include data relating to the location (e.g., a population of a city encompassing the location, a measure of pollution associated with the location, etc.). In some examples, sleep engine 200 may collect data, according to the systems and features described herein (e.g., at Steps 302 and 304 and elsewhere), for multiple users (e.g., unrelated users engaging with different instances of UE 102 within different instances of network 104). In these examples, database 108 may represent a repository of user data maintained for each of the multiple users.

In Step 306, sleep engine 200 can analyze the data (e.g., the user data) collected at Step 304. In embodiments in which user data is collected for multiple users, the analysis can include an analysis of each user's individual user data, an analysis of a deidentified aggregation of all of the users' user data, a demographic analysis (e.g., in which deidentified sleep data and/or sleep-relevant data is aggregated and analyzed for different demographics), and/or an analysis of how an individual user's data correlates with aggregated data. According to some embodiments, sleep engine 200 can implement any type of known or to be known computational analysis technique, algorithm, mechanism or technology to analyze the collected user data from Step 306.

In some embodiments, sleep engine 200 may include a specific trained artificial intelligence/machine learning model (AI/ML), a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, sleep engine 200 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, and the like. By way of a non-limiting example, engine 200 can implement an XGBoost algorithm for regression and/or classification to analyze the user data, as discussed herein.

According to some embodiments, the AI/ML computational analysis algorithms implemented can be applied and/or executed in a time-based manner, in that collected user data for specific time periods can be allocated to such time periods so as to determine patterns of activity (or non-activity) according to a criteria. For example, sleep engine 200 can execute a Bayesian determination for a predetermined time span, at preset intervals (e.g., a 24-hour time span and/or 8-hour time span, for example), so as to segment the day according to applicable patterns, which can be leveraged to determine, derive, extract or otherwise activities/non-activities (e.g., in/around a location). In another example, the patterns can correspond to portions of the day that correspond to nighttime or sleep time (e.g., which can be based on sunrise and/or sunset at the user's location and/or a window of time designated by a user).

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
c. apply the trained model to process the newly received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In Step 308, based on the analysis from Step 306, sleep engine 200 can determine (e.g., generate or receive), for a user, a demographic of the user and/or a measure of the user's sleep (e.g., one or more of the measures of sleep, such as deep sleep duration, discussed above at Step 304). In examples in which user data is collected and analyzed for multiple users, sleep engine 200 may (e.g., upon determining measures of user sleep for each of the users) aggregate the measures of user sleep determined for each user into an aggregated measure of user sleep. In some examples, sleep engine 200 can generate an aggregated measure of user sleep for a population that includes each user for whom data is collected (e.g., collected in Steps 302 and 304). Additionally or alternatively, sleep engine 200 can generate an aggregated measure of user sleep for a population defined by a user demographic. Such a population may include users associated any detected demographic (e.g., one or more of the demographics discussed in Step 304). Examples of such a population may include, without limitation, a population of users of a particular age (e.g., users with an age that falls within a designated range), a population of users who share an occupation (e.g., users with a particular type of occupation, users with a specific occupation, users for work for a particular institution and/or type of institution, etc.), and/or a population of users of a particular weight and/or with a particular BMI score (e.g., with a weight and/or BMI score that falls within a determined range). Sleep engine 200 can select demographics around which to define a population in a variety of ways. In some examples, sleep engine 200 can select the demographics around which to define a population using one or more of the computational analysis techniques, algorithms, mechanisms or technologies described above at Step 306. In other examples, the demographics may be manually selected.

Sleep engine 200 can generate an aggregate measure of user sleep for users within a population (e.g., users who share a specified demographic) in a variety of ways. For example, sleep engine 200 can generate an average measure of user sleep within a population, a median measure of user sleep within a population, and/or a distribution of measures of user sleep within a population. As a specific example, sleep engine 200 may determine that, for a population defined as all users who live in a particular location (e.g., on a particular street and/or in a particular neighborhood, city, and/or country), the average duration of deep sleep is 45 minutes per night. In some examples, the aggregate measure may be generated using one or more of the computational analysis techniques, algorithms, mechanisms or technologies described above at Step 306.

In some examples in which an aggregate measure of user sleep is generated for a population (e.g., a population associated with a specified demographic), sleep engine 200 may (e.g., using one or more of the computational analysis techniques, algorithms, mechanisms or technologies described above at Step 306) determine, for a user determined to be associated with the demographic, a correlation between a measure of sleep generated for the user and the aggregate measure of sleep. The correlation can include and/or represent any qualitative and/or quantitative comparison between the user's measure of sleep and the measures of sleep generated for users within the population (e.g., the aggregate measure of sleep). In some examples, the correlation can include and/or represent (1) two numerical values (a first value corresponding to the user's measure of sleep and a second value corresponding to the aggregate measure of sleep) and/or a (2) relational value indicating a difference between the two numerical values. In certain embodiments, a measure of user sleep may be assigned to a category (e.g., good, adequate, or poor) and the correlation may include two categories (a first category assigned to the user's measure of sleep and a second category assigned to the aggregate measure of sleep). In one embodiment, the correlation may include a relative description (e.g., “better than average,” “below average,” “average,” etc.) that describes how the user's measure of sleep correlates (e.g., compares) with the aggregate measure of sleep.

In some examples, based on the analysis from Step 306, sleep engine 200 can determine one or more features associated with a user. The features determined by sleep engine 200 may represent any type or form of computer-detectable feature (e.g., collected and/or derived in Step 304). In some examples, sleep engine 200 can determine an environmental feature (e.g., a location associated with the user, such as a location where the user lives and/or works, and/or a feature of the location). As a specific example, sleep engine 200 can determine, as associated with the user, a geographic area (e.g., a particular city) and/or a type of geographic area (e.g., an urban area), an altitude of a geographic area, a measure of pollution at a geographic area (e.g., air pollution, water pollution, light pollution, etc.), a population size of a geographic area, etc. In some examples, sleep engine 200 can determine a biometric feature (e.g., a weight of the user, a blood oxygen level of the user, a temperature of the user, a heartrate of the user, a blood pressure of the user, etc.). In some examples, sleep engine 200 can determine, as a feature, a pattern of activity (e.g., a set of patterns for the user and/or patterns for a location of the user). According to some embodiments, the determined patterns are based on the computational AI/ML analysis performed via sleep engine 200, as discussed above. In some embodiments, the set of patterns can correspond to, but are not limited to, types of events, types of detected activity, a time of day, a date, type of user, duration, amount of activity, quantity of activities, sublocations within the location (e.g., rooms in the house, for example), and the like, or some combination thereof. Accordingly, the patterns can be specific to a user, and/or specific to the location (e.g., or room within a location—for example, the bedroom of the location).

In some examples, sleep engine 200 can (e.g., based on the computational AI/ML analysis discussed in Step 306) identify a subset of users within a population (e.g., a general population and/or a population that shares a specified demographic) who have a measure of user sleep that falls below a threshold value. As a specific example, sleep engine 200 can identify, from a population of users who are between the ages of 30 and 40, a subset of users who average less than a threshold amount of deep sleep. In some examples, the threshold amount may be static (e.g., a threshold corresponding to a value associated with healthy sleep). In other examples, the threshold amount may be based on population data (e.g., the threshold amount may be set as the average aggregate sleep measure for the relevant population).

Additionally, sleep engine 200 can determined (e.g., based on the computational AI/ML analysis discussed in Step 306 as an output from a trained model) a feature that is associated with users within the subset (e.g., a feature associated with more than a threshold number and/or percentage of the users within the subset). Sleep engine 200 can identify any type or form of feature associated with users with the subset (e.g., an environmental, biometric, and/or pattern of activity metric as described previously in Step 308). In some examples, the determined feature may represent a feature that (1) is associated with users within the subset and (2) is not associated with users in an additional subset of users within the population who have a measure of user sleep that falls above the threshold value. Returning to the specific example in which the population represents a population of users who are between the ages of 30 and 40, the determined feature may represent a feature that (1) is associated with users, between the ages of 30 and 40, who average less than the threshold amount of deep sleep and that (2) is not associated with users, between the ages of 30 and 40, who average greater than the threshold amount of deep sleep. Relatedly, the determined feature may represent a feature that (1) is not associated with users within the subset and (2) is associated with users within the additional subset of users.

In Step 310, sleep engine 200 can store the determined information (e.g., the determined correlation and/or feature) in database 108, in a similar manner as discussed above. According to some embodiments, Step 310 can involve creating a data structure associated with each determined correlation and/or feature, whereby each data structure can be stored in a proper storage location associated with an identifier of the user/location, as discussed above. In some embodiments, the data structure can be configured with header (or metadata) that identifies a user and/or the location, a demographic and/or population used for the correlation, and/or a time period/interval of analysis (as discussed above); and the remaining portion of the structure providing the data of the activity/non-activity and/or status of entry-points during such sequence(s). In some embodiments, the data structure for a pattern can be relational, in that the events of a pattern can be sequentially ordered, and/or weighted so that the order corresponds to events with more or less activity.

In some embodiments, the structure of the data structure for a correlation and/or feature can enable a more computationally efficient (e.g., faster) search of the correlation and/or feature. In some embodiments, the data structures of correlations can be, but are not limited to, files, arrays, lists, binary, heaps, hashes, tables, trees, and the like, and/or any other type of known or to be known tangible, storable digital asset, item and/or object.

According to some embodiments, the user data can be identified and analyzed in a raw format, whereby upon a determination of the pattern, the data can be compiled into refined data (e.g., a format capable of being stored in and read from database 108). Thus, in some embodiments, Step 310 can involve the creation and/or modification (e.g., transformation) of the user data into a storable format.

In Step 312, sleep engine 200 can output a correlation, determined for a user in Step 308, to the user (e.g., by communicating the correlation to the user over a network such as network 104). The correlation may be output to the user in a variety of ways. For example, the correlation can be displayed on a device of the user (e.g., in the form of an application notification, electronic message, and the like). In one such example, sleep engine 200 can generate a user interface display that includes the correlation and/or can trigger a user device of the user (e.g., operating within UE 102) to present the correlation (e.g., via the user interface display) via a display element of the user device. In some examples, the user interface display can be configured to present the correlation to the user as part of an explanation of how the user compares with other users who share a particular demographic with the user. As a specific example, the user interface display may include text such as “You had twenty-five minutes of deep sleep last night. This is twenty minutes lower than the average for an engineering student.” “You slept for 7 hours and 33 minutes last night, the median amount of sleep in your neighborhood is 7 hours and 45 minutes,” “Your sleep is above average for a health care worker,” or “You appear to have a moderately-low sleep quality. This is the most common categorization for employees at your institution.”

In some examples (as mentioned previously), sleep engine 200 can determine, for a user within a certain population, that a measure of the user's sleep falls below a threshold and/or that the user is associated with a feature determined to be associated with a subset of users within the population who have a measure of sleep that falls below the threshold. In some such examples, in response to this determination, sleep engine 200 can determine and/or compile a sleep optimization recommendation based on the feature. Examples of sleep optimization recommendations can include, without limitation, screen time limits, Internet traffic limits, television limits, preferred temperatures in the location, lighting instructions, recommendations for amount of blankets on the bed as per the user's ideal sleep temperature (against the indoor temperature and outside climate), amount of pillows (e.g., head or leg angle to induce ideal blood flow for the user during sleep, a time to cut off eating, a preferred time to shower, and the like, or some combination thereof.

In examples in which a user interface display (e.g., including the correlation) is presented to the user, sleep engine 200 can configure the user interface display to present the sleep optimization recommendation (e.g., together with an explanation of how the feature was determined). As a specific example, the user interface display can further include text such as “You might improve your sleep by cutting down the screen time before bed. You average twenty minutes of screen time on your smart phone in the hour before sleeping. Engineering students who average between ten and twenty minutes of screen time in the hour before sleeping have, on average, ten fewer minutes of deep sleep than engineering students who average between zero and ten minutes of screen time in the hours before bed.” As another example, the user interface display can further include text such as “Have you ever considered moving out of state? Users with your occupation who live in X state sleep an average of 45 minutes more each night that users with your occupation living in your state.” In some embodiments, sleep optimization recommendations can be compiled as a data structure and stored in database 108, in a similar manner as discussed above in Step 310.

In addition, or as an alternative to, providing a sleep optimization recommendation (e.g., as a set of instructions that can be implemented and/or executed so as to enable a user to engage in a more restful sleep), in certain examples, sleep engine 200 can control the digital environment for the user according to the sleep optimization recommendation. As a specific example, the sleep optimization recommendation can include a recommendation to keep the lights at a lower level in the hour before sleep and sleep engine 200 can automatically trigger lights in the user's home to dim at a time designated as the hour before bed.

As shown in the figure, in some embodiments, Client device 400 includes a processing unit (CPU) 422 in communication with a mass memory 430 via a bus 424. Client device 400 also includes a power supply 426, one or more network interfaces 450, an audio interface 452, a display 454, a keypad 456, an illuminator 458, an input/output interface 460, a haptic interface 462, an optional global positioning systems (GPS) receiver 464 and a camera(s) or other optical, thermal or electromagnetic sensors 466. Device 400 can include one camera/sensor 466, or a plurality of cameras/sensors 466, as understood by those of skill in the art. Power supply 426 provides power to Client device 400.

Client device 400 may optionally communicate with a base station (not shown), or directly with another computing device. In some embodiments, network interface 450 is sometimes known as a transceiver, transceiving device, or network interface card (NIC).

Audio interface 452 is arranged to produce and receive audio signals such as the sound of a human voice in some embodiments. Display 454 may be a liquid crystal display (LCD), gas plasma, light emitting diode (LED), or any other type of display used with a computing device. Display 454 may also include a touch sensitive screen arranged to receive input from an object such as a stylus or a digit from a human hand.

Keypad 456 may include any input device arranged to receive input from a user. Illuminator 458 may provide a status indication and/or provide light.

Client device 400 also includes input/output interface 460 for communicating with external. Input/output interface 460 can utilize one or more communication technologies, such as USB, infrared, Bluetooth™, or the like in some embodiments. Haptic interface 462 is arranged to provide tactile feedback to a user of the client device.

Optional GPS transceiver 464 can determine the physical coordinates of Client device 400 on the surface of the Earth, which typically outputs a location as latitude and longitude values. GPS transceiver 464 can also employ other geo-positioning mechanisms, including, but not limited to, triangulation, assisted GPS (AGPS), E-OTD, CI, SAI, ETA, BSS or the like, to further determine the physical location of client device 400 on the surface of the Earth. In one embodiment, however, Client device may through other components, provide other information that may be employed to determine a physical location of the device, including for example, a MAC address, Internet Protocol (IP) address, or the like.

Mass memory 430 includes a RAM 432, a ROM 434, and other storage means. Mass memory 430 illustrates another example of computer storage media for storage of information such as computer readable instructions, data structures, program modules or other data. Mass memory 430 stores a basic input/output system (“BIOS”) 440 for controlling low-level operation of Client device 400. The mass memory also stores an operating system 441 for controlling the operation of Client device 400.

Memory 430 further includes one or more data stores, which can be utilized by Client device 400 to store, among other things, applications 442 and/or other information or data. For example, data stores may be employed to store information that describes various capabilities of Client device 400. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 500.

Applications 442 may include computer executable instructions which, when executed by Client device 400, transmit, receive, and/or otherwise process audio, video, images, and enable telecommunication with a server and/or another user of another client device. Applications 442 may further include a client that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A computer-implemented method comprising:
generating, by a device for a user, a measure of the user's sleep based on data collected by a sensor of a sleep-tracking device;
determining, by the device, a demographic of the user and a correlation between the measure of the user's sleep and measures of user sleep generated for other users associated with the demographic; and
communicating, over a network, the correlation to the user via a digital display presented via a display element of a user device.

2. The method of claim 1, wherein the demographic comprises at least one of:
an age;
a gender;
a weight;
a BMI score;
an occupation;
a level of education;
a family status;
an ethnicity;
a location; and
a culture.

3. The method of claim 1, further comprising triggering the sensor to collect the sleep data.

4. The method of claim 1, wherein the measure of the user's sleep and the measures of user sleep generated for the other users comprise a measure of at least one of sleep latency, total sleep duration, deep sleep duration, light sleep duration, sleep restlessness, or nighttime awakenings.

5. The method of claim 1, further comprising:
identifying a subset of the users associated with the demographic whose measure of user sleep falls below a threshold value;
receiving, as an output from a trained model, a feature associated with the users within the subset of users whose measure of user sleep falls below the threshold value; and
communicating, over the network, the feature to the user via the digital display as part of a suggestion for improving the measure of the user's sleep.

6. The method of claim 5, wherein communicating the feature to the user comprises communicating the feature in response to at least one of:
determining that the measure of the user's sleep falls below the threshold value; and
determining that the user is associated with the feature.

7. The method of claim 5, further comprising:
for each particular user within the subset of users whose measure of user sleep falls below the threshold value, identifying one or more devices within a location associated the particular user;
collecting data from the identified devices; and
inputting the data collected from the identified devices to the trained model, wherein the feature is generated by the trained model as the output based on the inputted data.

8. The method of claim 5, wherein the feature comprises an environmental feature.

9. The method of claim 8, wherein the environmental feature comprises at least one of:
a geographic area associated with the user;
an altitude of a geographic area associated with the user;
a measure of pollution at a geographic area associated with the user; and
a population size of a geographic area associated with the user.

10. The method of claim 5, wherein the feature comprises a pattern of activity.

11. The method of claim 10, wherein the pattern of activity comprises at least one of:
a pattern of screen usage; and
a pattern of movement.

12. The method of claim 10, wherein the pattern of activity is determined for each particular user within the subset in response to:
identifying a set of devices associated with a location of the particular user;
collecting and analyzing data from each of the set of devices; and
in response to analyzing the data from each of the set of devices, determining the pattern of activity for the particular user.

13. The method of claim 12, wherein the set of devices corresponds to devices comprising sensor capabilities for tracking movements and information about the particular user at the location.

14. The method of claim 5, wherein the feature comprises a biometric feature.

15. The method of claim 14, wherein the biometric feature comprises at least one of:
a weight of the user;
a blood oxygen level of the user;
a temperature of the user;
a heartrate of the user; and
a blood pressure of the user.

16. The method of claim 1, wherein the sleep-tracking device comprises a wearable device.

17. A system comprising:
a physical processor; and
physical memory comprising computer-executable instructions that, when executed by the physical processor, cause the physical processor to:
generate, for a user, a measure of the user's sleep based on data collected by a sensor of a sleep-tracking device;
determine a demographic of the user and a correlation between the measure of the user's sleep and measures of user sleep generated for other users associated with the demographic; and
communicate, over a network, the correlation to the user via a digital display presented via a display element of a user device.

18. The system of claim 17, wherein the demographic comprises at least one of:
an age;
a gender;
a weight;
a BMI score;
an occupation;
a level of education;
a family status;
an ethnicity;
a location; and
a culture.

19. The system of claim 17, wherein the computer-executable instructions further cause the physical processor to trigger the sensor to collect the sleep data.

20. A non-transitory computer-readable storage medium tangibly encoded with computer-executable instructions that, when executed by a device, perform a method comprising:
generating, for a user, a measure of the user's sleep based on data collected by a sensor of a sleep-tracking device;
determining a demographic of the user and a correlation between the measure of the user's sleep and measures of user sleep generated for other users associated with the demographic; and
communicating, over a network, the correlation to the user via a digital display presented via a display element of a user device.
